(19) United States
(12) Patent Application Publication
(10) Pub. No.: US 2014/0127335 A1
(43) Pub. Date: May 8, 2014

(54) FRAGRANCE PRIMER

(71) Applicants: Heath Miller, New York, NY (US); Adam Hopkins, New York, NY (US)

(72) Inventors: Heath Miller, New York, NY (US); Adam Hopkins, New York, NY (US)

(73) Assignee: Concrete & Canvas, LLC, New York, NY (US)

(21) Appl. No.: 14/074,382

(22) Filed: Nov. 7, 2013

Related U.S. Application Data
(60) Provisional application No. 61/723,868, filed on Nov. 8, 2012.

(51) Int. Cl. A61K 8/97; A61Q 13/00; A61K 8/81; A61K 8/67; A61K 8/96; A61K 8/34

(52) U.S. Cl. CPC A61K 8/97; A61K 8/96; A61K 8/34; A61K 8/8158; A61K 8/8152; A61K 8/678; A61Q 13/00
USPC 424/758

(57) ABSTRACT

A fragrance primer composition may include an astringent, a humectant, a film former, a solvent, a vitamin and at least three botanical products. The components may be ethyl alcohol, butylene glycol, acrylates/acrylamide copolymer, water, tocopheryl acetate, cucumis sativus fruit extract, chamomilla recutita flower/leaf extract, and aloe barbadensis leaf juice respectively. The fragrance primer composition may be applied as a surface barrier to a user's skin from a fragrance, perfume, and/or cologne.

### CROSS-REFERENCE TO RELATED APPLICATION

This application claims the benefit of U.S. provisional application No. 61/723,868, filed Nov. 8, 2012, the contents of which are herein incorporated by reference.

### BACKGROUND OF THE INVENTION

The present invention relates to fragrances, perfumes, and/or colognes and, more particularly, to a fragrance primer.

Fragrances, perfumes and colognes are mixtures of essential oils or aroma compounds, fixatives and solvents used to give humans, animals, objects, and living spaces a pleasant scent. Fragrances, perfumes and colognes have been known to exist in some of the earliest human civilizations.

Fragrances, perfumes and colognes can be created through fragrance extraction of aromatic compounds of raw materials, using methods such as distillation, solvent extraction, expression or enfleurage. The results of the extracts can be essential oils, absolutes, concretes, or butters, depending on the amount of waxes in the extracted product.

Over the years, many types of fragrances, perfumes and colognes have been used, developed and/or suggested. These conventional fragrances, perfumes and/or colognes have met with varying degrees of success. However, many fragrances, perfumes, and/or colognes are not effective and the scent or smell changes once they make contact with human skin. Furthermore, many fragrances, perfumes, and/or colognes are expensive and do not last very long once applied.

When sprayed or applied onto skin, the scent of fragrances, perfumes, and/or colognes combine with the user's skin, natural oils, and natural pH, which together in combination change the inherent scent or smell of the fragrances, perfumes, and/or colognes and alters the fragrance, perfume, and/or cologne designer's intended scent or smell. Moreover, when fragrances, perfumes, and colognes are sprayed or applied onto skin, they often are absorbed quickly and wear off in a matter of hours. After a few hours, the scent can no longer be detected.

Conventional cosmetic primers usually comprise of a cream or lotion which is applied before another cosmetic to improve coverage and lengthen the amount of time the cosmetic lasts on the face, head, or other portions of the body. Much like primers used on a canvas for a painting, conventional cosmetic primers provide a base from which other cosmetics can be applied so the cosmetic adheres properly to the skin and the integrity of the cosmetic's actual color remains intact. Conventional cosmetic primers, however, are not effective with fragrances perfumes and colognes.

As can be seen, there is a need for a fragrance primer that may allow a fragrance, perfume, and/or cologne to keep a consistent scent for a longer period of time once applied.

### SUMMARY OF THE INVENTION

In one aspect of the present invention, a fragrance primer composition comprises: an astringent; a humectant; a film former; a solvent; a vitamin; and at least three botanical products.

In another aspect of the present invention, a method for priming a fragrance, perfume, and/or cologne comprises: providing a composition comprising an astringent; a humectant; a film former; a solvent; a vitamin; and at least three botanical products; and applying the composition to a surface of a body prior to the application of a fragrance, perfume, and/or cologne.

These and other features, aspects and advantages of the present invention will become better understood with reference to the following drawings, description and claims.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention may provide a fragrance primer composition that may include an astringent, a humectant, a film former, a solvent, a vitamin and at least three botanical products. The components may be ethyl alcohol, butylene glycol, acrylates/acrylamide copolymer, water, tocopheryl acetate, cucumis sativus fruit extract, chamomilla recutita flower/leaf extract, and aloe barbadensis leaf juice respectively. The fragrance primer composition may be applied as a surface barrier to a user's skin from a fragrance, perfume, and/or cologne.

The fragrance primer may be applied to an area of the skin of a user before a fragrance, perfume, or cologne may be sprayed or otherwise applied on the fragrance primer to improve coverage of the fragrance, perfume, or cologne, lengthen the amount of time the fragrance, perfume, and/or cologne lasts, and keep the integrity of the scent intact.

In certain embodiments, the fragrance primer may be applied by, but not limited to, spray. In certain embodiments, the fragrance primer may be used by spraying a layer of the fragrance primer onto areas where a user normally uses and plans to apply their fragrance, perfume, and/or cologne, including, but not limited to, a user's wrist, neck, ears, face, head, hair, chest, legs or other portions of the body. After the fragrance primer dries, the user may spray or otherwise apply their fragrance, perfume, and/or cologne onto the area previously sprayed onto by the fragrance primer.

The fragrance primer provides a surface barrier to the skin of the user from the fragrance, perfume and/or cologne so that the scent of the fragrance, perfume, and/or cologne may not combine with the user's skin, natural oils, or natural pH. The surface barrier created by the fragrance primer between the user's skin and the fragrance, perfume, and/or cologne may reduce the amount of absorption of the fragrance, perfume, and/or cologne into the user's skin. This surface barrier allows the scent of the fragrance, perfume, and/or cologne to remain closer to what the user may smell while the product is still in a bottle, and to what a designer of the fragrance, perfume, and/of cologne may have intended to be the scent of the fragrance, perfume, and/or cologne.

In certain embodiments, the surface barrier created by the fragrance primer may allow the scent of the fragrance, perfume, and/or cologne to stay on the surface and therefore last longer, throughout the day and evening. The scent of the fragrance, perfume, and/or cologne may last longer with the fragrance primer, than with just the fragrance, perfume, and/or cologne in contact with the user's skin directly.

In certain embodiments, the fragrance primer may be applied to the skin of the user by, but not limited to, spray, concrete solid wax, pomade, cream, lotion, oil poured directly on the skin and the like. The amount of coverage may vary based on the amount of the fragrance primer that is used. The application of the fragrance primer may allow for fewer applications of the fragrance, perfume, and/or cologne which may reduce costs for the same intended results.

In certain embodiments, the fragrance primer may also be used as a primer for scented makeup, including, but not limited to, foundation, eye shadow, lipstick, blush, and other cosmetics.

The present invention may include a composition. The composition may include the following ingredients; ethyl alcohol; butylene glycol; acrylates/acrylamide copolymer; water; tocopheryl acetate; cucumis sativus fruit extract; chamomilla recutita flower/leaf extract; and aloe barbadensis leaf juice.

In certain embodiments, the fragrance primer may include the following percent ranges: approximately 30-100% ethyl alcohol; approximately 10-30% butylene glycol; approximately 1-3% acrylates/acrylamide copolymer; approximately 0.01-0.10% water; approximately 0.01-0.10% tocopheryl acetate; approximately 0.01-0.10% cucumis sativus fruit extract; approximately 0.01-0.10% chamomilla recutita flower/leaf extract; and approximately 0.01-0.10% aloe barbadensis leaf juice.

In certain embodiments, the fragrance primer may include the following percents in weight: approximately 68.75% ethyl alcohol; approximately 29% butylene glycol; approximately 2% acrylates/acrylamide copolymer; approximately 0.05% water; approximately 0.05% tocopheryl acetate; approximately 0.05% cucumis sativus fruit extract; approximately 0.05% chamomilla recutita flower/leaf extract; and approximately 0.05% aloe barbadensis leaf juice

In certain embodiments, the ethyl alcohol may be used as an astringent; butylene glycol may be used as a humectant; acrylates/acrylamide copolymer may be used as a film former; water may be used as a solvent; tocopheryl acetate may be used as a vitamin; and cucumis sativus fruit extract, chamomilla recutita flower/leaf extract, and aloe barbadensis leaf juice may be used as botanical items. In certain embodiments, the fragrance primer may be free of scent, all natural and easy to use.

It should be understood, of course, that the foregoing relates to exemplary embodiments of the invention and that modifications may be made without departing from the spirit and scope of the invention as set forth in the following claims.

What is claimed is:

1. A fragrance primer composition comprising:
an astringent; a humectant; a film former; a solvent; a vitamin; and at least three botanical products.

2. The fragrance primer composition of claim 1, wherein the astringent is ethyl alcohol, wherein the humectants is butylene glycol, wherein the film former is acrylates/acrylamide copolymer, wherein the solvent is water, wherein the vitamin is tocopheryl acetate, wherein the at least three botanical products comprises cucumis sativus fruit extract, chamomilla recutita flower/leaf extract and aloe barbadensis leaf juice.

3. The fragrance primer composition of claim 1, in weight percent, from about 30 to about 100 percent ethyl alcohol, from about 10 to about 30 percent butylene glycol, from about 1 to about 3 percent acrylates/acrylamide copolymer, from about 0.01 to about 0.10 percent water, from about 0.01 to about 0.10 percent tocopheryl acetate, from about 0.01 to about 0.10 percent cucumis sativus fruit extract, from about 0.01 to about 0.10 percent chamomilla recutita flower/leaf extract, and from about 0.01 to about 0.10 aloe barbadensis leaf juice.

4. The fragrance primer composition of claim 1, in weight percent, approximately 68.75 percent ethyl alcohol, approximately 29 percent butylenes glycol, approximately 2 percent acrylates/acrylamide copolymer, approximately 0.05 percent water, approximately 0.05 percent tocopheryl acetate, approximately 0.05 percent cucumis sativus fruit extract, approximately 0.05 percent chamomilla recutita flower/leaf extract, and approximately 0.05 aloe barbadensis leaf juice

5. A method for priming a fragrance, perfume, and/or cologne comprising:
providing a composition comprising an astringent; a humectant; a film former; a solvent; a vitamin; and at least three botanical products; and
applying the composition to a surface of a body prior to the application of a fragrance, perfume, and/or cologne.

6. The method of claim 5, wherein the surface of the body comprises at least one of the wrist, neck, face, head, hair, chest, and legs.
